(19) United States
(12) Patent Application Publication
(10) Pub. No.: US 2023/0277543 A1
(43) Pub. Date: Sep. 7, 2023

(54) COMPOSITIONS AND METHODS FOR TREATING DISEASES ASSOCIATED WITH ELEVATED ARGININE VASOPRESSIN LEVELS

(71) Applicant: The United States Government As Represented By The Department Of Veterans Affairs, Washington, DC (US)

(72) Inventors: Bellamkonda Kishore, Salt Lake City, UT (US); Noel Carlson, Salt Lake City, UT (US)

(21) Appl. No.: 18/175,630

(22) Filed: Feb. 28, 2023

Related U.S. Application Data
(60) Provisional application No. 63/315,237, filed on Mar. 1, 2022.

(51) Int. Cl. A61K 31/519; A61K 45/06

(52) U.S. Cl. CPC A61K 31/519; A61K 45/06

(57) ABSTRACT

Disclosed herein, are compositions comprising ticagrelor metabolites and methods of using the same to treat congestive heart failure, autosomal dominant polycystic kidney and liver diseases, cirrhosis of liver, cardiorenal syndrome, high altitude pulmonary hypertension, severe depression and autism.

### CROSS REFERENCE TO RELATED APPLICATIONS

This application claims the benefit of U.S. Provisional Application No. 63/315,237, filed Mar. 1, 2022. The content of this earlier filed application is hereby incorporated by reference herein in its entirety.

### BACKGROUND

Autosomal dominant polycystic kidney disease (ADPKD) is the most common inherited disease of the kidney, with a prevalence at birth ranging from 1 per 500 to 1000 people worldwide. ADPKD is caused by mutations in the PKD1 (85%) or PKD2 (15%) genes, which encode for polycystin-1 or polycystin-2 proteins, respectively. The hallmark of ADPKD is the formation of cysts in both kidneys, which gradually grow in size resulting in a decline of kidney function. By the age 55 years, about 50% of the ADPKD patients develop end-stage renal disease, which requires dialysis therapy or renal transplantation.

No specific therapy for ADPKD exists and management of ADPKD is limited to control of high blood pressure, and symptomatic treatment of complications. Current therapeutic approaches are directed to slowing the progression of cyst growth, but not without significant side effects.

### SUMMARY

Disclosed herein are method for inhibiting arginine vasopressin (AVP) production in a subject, the methods comprising administering a composition comprising an effective amount of AR-C124910XX or AR-C133913XX to the subject, thereby inhibiting AVP production in the subject.

Disclosed herein are methods for treating a kidney disease associated with elevated arginine vasopressin (AVP) levels in a subject, the methods comprising administering a composition comprising an effective amount of AR-C124910XX or AR-C133913XX to the subject, thereby decreasing AVP production in the subject.

Disclosed herein are methods for treating a liver disease associated with elevated arginine vasopressin (AVP) levels in a subject, the methods comprising administering a composition comprising an effective amount of AR-C124910XX or AR-C133913XX to the subject, thereby decreasing AVP production in the subject.

Disclosed herein are methods for lowering circulating levels of arginine vasopressin (AVP) in a subject, the methods comprising administering a composition comprising an effective amount of AR-C124910XX or AR-C133913XX to the subject, thereby lowering circulating levels of AVP in the subject.

Disclosed herein are methods of inhibiting cyst growth in a kidney in a subject suffering from a kidney disease associated with elevated arginine vasopressin (AVP), the methods comprising administering a composition comprising an effective amount of AR-C124910XX or AR-C133913XX to the subject, thereby inhibiting cyst grown in the kidney of the subject.

Disclosed herein are methods for treating congestive heart failure in a subject, the methods comprising administering a composition comprising an effective amount of AR-C124910XX or AR-C133913XX to the subject, thereby decreasing AVP production in the subject.

Disclosed herein are methods for treating cirrhosis of the liver in a subject, the methods comprising administering a composition comprising an effective amount of AR-C124910XX or AR-C133913XX to the subject, thereby decreasing AVP production in the subject.

Disclosed herein are method for treating cardiorenal syndrome in a subject, the methods comprising administering a composition comprising an effective amount of AR-C124910XX or AR-C133913XX to the subject, thereby decreasing AVP production in the subject.

Disclosed herein are methods for treating high altitude pulmonary hypertension in a subject, the methods comprising administering a composition comprising an effective amount of AR-C124910XX or AR-C133913XX to the subject, thereby decreasing AVP production in the subject.

Disclosed herein are methods for treating severe depression in a subject, the methods comprising administering a composition comprising an effective amount of AR-C124910XX or AR-C133913XX to the subject, thereby decreasing AVP production in the subject.

Disclosed herein are methods for treating autism in a subject, the methods comprising administering a composition comprising an effective amount of AR-C124910XX or AR-C133913XX to the subject, thereby decreasing AVP production in the subject.

Additional advantages of the disclosed method and compositions will be set forth in part in the description which follows, and in part will be understood from the description, or may be learned by practice of the disclosed method and compositions. The advantages of the disclosed method and compositions will be realized and attained by means of the elements and combinations particularly pointed out in the appended claims. It is to be understood that both the foregoing general description and the following detailed description are exemplary and explanatory only and are not restrictive of the invention as claimed.

### BRIEF DESCRIPTION OF THE DRAWINGS

The accompanying drawings, which are incorporated in and constitute a part of this specification, illustrate several embodiments of the disclosed method and compositions and together with the description, serve to explain the principles of the disclosed method and compositions.

FIG. 1 shows the structure of AR-C124910XX.

FIG. 2 shows the structure of AR-C133913XX.

FIG. 3 shows the mRNA expression of AVP in the primary cultures of rat hypothalamic cells exposed to PSB-0739 or a combination of PSB-0739 and 2 MeS-ADP for 24 hours.

### DETAILED DESCRIPTION

The present disclosure can be understood more readily by reference to the following detailed description of the invention, the figures and the examples included herein.

Before the present compositions and methods are disclosed and described, it is to be understood that they are not limited to specific synthetic methods unless otherwise specified, or to particular reagents unless otherwise specified, as such may, of course, vary. It is also to be understood that the terminology used herein is for the purpose of describing particular aspects only and is not intended to be limiting. Although any methods and materials similar or equivalent to those described herein can be used in the practice or testing of the present invention, example methods and materials are now described.

Moreover, it is to be understood that unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is in no way intended that an order be inferred, in any respect. This holds for any possible non-express basis for interpretation, including matters of logic with respect to arrangement of steps or operational flow, plain meaning derived from grammatical organization or punctuation, and the number or type of aspects described in the specification.

All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present invention is not entitled to antedate such publication by virtue of prior invention. Further, the dates of publication provided herein can be different from the actual publication dates, which can require independent confirmation.

### Definitions

As used in the specification and the appended claims, the singular forms “a,” “an” and “the” include plural referents unless the context clearly dictates otherwise.

The word “or” as used herein means any one member of a particular list and also includes any combination of members of that list.

Throughout the description and claims of this specification, the word “comprise” and variations of the word, such as “comprising” and “comprises,” means “including but not limited to,” and is not intended to exclude, for example, other additives, components, integers or steps. In particular, in methods stated as comprising one or more steps or operations it is specifically contemplated that each step comprises what is listed (unless that step includes a limiting term such as “consisting of”), meaning that each step is not intended to exclude, for example, other additives, components, integers or steps that are not listed in the step.

Ranges can be expressed herein as from “about” or “approximately” one particular value, and/or to “about” or “approximately” another particular value. When such a range is expressed, a further aspect includes from the one particular value and/or to the other particular value. Similarly, when values are expressed as approximations, by use of the antecedent “about,” or “approximately,” it will be understood that the particular value forms a further aspect. It will be further understood that the endpoints of each of the ranges are significant both in relation to the other endpoint and independently of the other endpoint. It is also understood that there are a number of values disclosed herein and that each value is also herein disclosed as “about” that particular value in addition to the value itself. For example, if the value “10” is disclosed, then “about 10” is also disclosed. It is also understood that each unit between two particular units is also disclosed. For example, if 10 and 15 are disclosed, then 11, 12, 13, and 14 are also disclosed.

As used herein, the terms “optional” or “optionally” mean that the subsequently described event or circumstance may or may not occur and that the description includes instances where said event or circumstance occurs and instances where it does not.

Other than in the operating examples, or where otherwise indicated, numbers expressing quantities of ingredients or reaction conditions used herein should be understood as modified in all instances by the term “about.” The term “about” when used in connection with percentages can mean a range of +1-10%.

As used herein, the term “subject” refers to the target of administration, e.g., a human. Thus, the subject of the disclosed methods can be a vertebrate, such as a mammal, a fish, a bird, a reptile, or an amphibian. The term “subject” also includes domesticated animals (e.g., cats, dogs, etc.), livestock (e.g., cattle, horses, pigs, sheep, goats, etc.), and laboratory animals (e.g., mouse, rabbit, rat, guinea pig, fruit fly, etc.). In some aspects, a subject is a mammal. In some aspects, the subject is a human. The term does not denote a particular age or sex. Thus, adult, child, adolescent and newborn subjects, as well as fetuses, whether male or female, are intended to be covered.

As used herein, the term “patient” refers to a subject afflicted with a disease, disorder or condition. The term “patient” includes human and veterinary subjects. In some aspects of the disclosed methods, the “patient” has been diagnosed with a need for treatment for preventing or treating autosomal dominant polycystic kidney disease, autosomal dominant polycystic liver dis-ease, congestive heart failure, cirrhosis of the liver, cardiorenal syndrome, high altitude pulmonary hypertension, severe depression and autism, such as, for example, prior to the administering step.

The term “therapeutic” refers to a composition that treats a disease. For example, the therapeutics disclosed herein are compositions that treat autosomal dominant polycystic kidney disease, autosomal dominant polycystic liver disease, congestive heart failure, cirrhosis of the liver, cardiorenal syndrome, high altitude pulmonary hypertension, severe depression and autism.

As used herein, the term “therapeutically effective amount” means an amount of a therapeutic, prophylactic, and/or diagnostic agent (e.g., AR-C124910XX or AR-C133913XX) that is sufficient, when administered to a subject suffering from or susceptible to a disease, disorder, and/or condition, to treat, alleviate, ameliorate, relieve, alleviate symptoms of, prevent, delay onset of, inhibit progression of, reduce severity of, and/or reduce incidence of the disease, disorder, and/or condition. In some instances, a therapeutically effective amount is an amount of a therapeutic that provides a therapeutic benefit to an individual.

As used herein, the term “treating” refers to partially or completely alleviating, ameliorating, relieving, delaying onset of, inhibiting or slowing progression of, reducing severity of, and/or reducing incidence of one or more symptoms or features of a particular disease, disorder, and/or condition. For example, “treating” a kidney disease, e.g., autosomal dominant polycystic kidney disease, may refer to slowing down the progression of the disease, reducing the number or size of cysts, and/or reducing the size of the kidneys. Treatment can be administered to a subject who does not exhibit signs of a disease, disorder, and/or condition and/or to a subject who exhibits only early signs of a disease, disorder, and/or condition for the purpose of decreasing the risk of developing pathology associated with the disease, disorder, and/or condition. In some aspects, “treating” a disease or a condition can mean inhibiting or reducing AVP production or reducing circulating levels of AVP.

As used herein, the term “derivative” refers to a compound having a structure derived from the structure of a parent compound (e.g., AR-C124910XX or AR-C133913XX) and whose structure is sufficiently similar to those disclosed herein and based upon that similarity, would be expected by one skilled in the art to exhibit the same or similar activities and utilities as AR-C124910XX or AR-C133913XX, or to induce, as a precursor, the same or similar activities and utilities as AR-C124910XX or AR-C133913XX. Exemplary derivatives include salts, esters, amides, salts of esters or amides, and N-oxides of a parent compound such as AR-C124910XX or AR-C133913XX.

As used herein, the term “production of arginine vasopressin or AVP” refers to or encompasses transcription of AVP gene, processing of the mRNA or translation or post-translational modification or storage or secretion of a combination of one or more of these.

Disclosed herein are compositions comprising AR-C124910XX, AR-C133913XX, derivatives and combinations of AR-C124910XX or AR-C133913XX and methods of using the compositions for treating diseases and conditions associated with increased circulating levels of arginine vasopressin (AVP). Diseases and conditions associated with increased circulating levels of AVP include, but are not limited to, autosomal dominant polycystic kidney disease (ADPKD), dilutional hyponatremia, congestive heart failure, cirrhosis of liver, cardiorenal syndrome, diabetic ketoacidosis, and high altitude pulmonary hypertension.

Autosomal dominant polycystic kidney disease (ADPKD) is the most common inherited disease of the kidney, with a prevalence ranging from 1 per 500 to 1,000 people worldwide. It is usually caused by mutations in the PKD1 (85%) or PKD2 (15%) genes, which encode for polycystin-1 or polycystin-2 proteins, respectively. The hallmark of ADPKD is the formation of cysts in both kidneys, which are filled with fluid. Over time, the number and size of the cysts gradually increase, replacing much of the normal structures of the kidney, resulting in chronic kidney disease, and renal failure. By the age 55, about 50% of the ADPKD patients develop end-stage renal disease (ESRD), which requires dialysis therapy or renal transplantation. Despite the magnitude of its pathophysiology and clinical outcome resulting in renal failure, no specific cure for ADPKD exists. Management of ADPKD is limited to control of high blood pressure, and symptomatic treatment of complications. Three different approaches are currently being tested for ADPKD: 1) suppression of cell proliferation by the use of inhibitors of mTOR (mechanistic target of rapamycin) activity (e.g., sirolimus and everolimus); 2) reduction of the activity of the tubular epithelium lining the cysts and thereby shrinking the cysts by the use of somatostatin analogues (e.g., octreotide and lanreotide); and 3) vasopressin V2 receptor antagonism with reduction in cyclic AMP (cAMP) production (e.g., tolvaptan) (Chang and Ong, J Clin Pharmacol 76:524-535, 2013). The rationale for the last approach is that AVP or the anti-diuretic hormone (ADH), produced by the hypothalamus in the brain, is a major stimulus for production of cAMP in the kidney collecting ducts, the predominant sites for the origin of cysts in the kidney. cAMP promotes cyst growth. Incidentally, the circulating levels of AVP are increased in human ADPKD and in animal models of ADPKD, where it has been ascertained (Tones, Clin J Am Soc Nephrol 3:1212-128, 2008). This may be due to compensatory increase in the production of AVP by the hypothalamus in response to reduced concentrating capacity of the polycystic kidneys. Thus, the AVP-V2 receptor-cAMP axis plays an important role in ADPKD progression. Accordingly, in pre-clinical studies in rodent models of ADPKD, vasopressin V2 receptor antagonists (OPC-31260 and tolvaptan) consistently inhibited cystogenesis by reducing cAMP production in the kidney (Gattone II et al, Nature Med 9:1323-1326, 2003; and Wang et al, J Am Soc Nephrol 16:846-851, 2005). In subsequent clinical trials, the efficacy of tolvaptan for the treatment of ADPKD was established (Tones, Semin Nephrol 28:306-317, 2008, PMID: 1851909). Nevertheless, the adverse effects of blocking vasopressin V2 receptor are significant, such as severe polyuria, nocturia, thirst and dry mouth in a vast majority of patients. Other common side effects of tolvaptan reported are constipation, loss of appetite, dry skin, nausea, vomiting and a potential for acute liver failure (Baur and Meaney, Pharmacother 34:605-616, 2014, PMID: 24706579). These and other factors limit the use of tolvaptan to ADPKD patients with rapidly declining renal function. Hence, there is an urgent need for other therapies to treat ADPKD patients.

Using ticagrelor metabolites (e.g., AR-C124910XX or AR-C133913XX) over the approach of selective blockade of V2 receptor by tolvaptan, which is approved by the FDA for use in patients with rapidly progressing ADPKD to reduce rate of decline in renal function has significant advantages. It has been shown that chronic blockade of vasopressin V2 receptor by tolvaptan results in increased endogenous production of AVP. Since AVP interacts with its two other receptors, namely V1a (in blood vessels) and V1b (in liver), the effects of chronically increased AVP levels through these two V1 receptor subtypes is not known. In contrast, the administration of ticagrelor decreases AVP levels in a dose-dependent fashion, and so the V1a and V1b receptors are not overstimulated. Thus, ticagrelor and its metabolites AR-C124910XX or AR-C133913XX may be on a different platform as compared to selective blockade of vasopressin V2 receptor by drugs such as tolvaptan.

As compared to the currently available alternative for reduction in the activity of AVP and production of cAMP in the kidney, i.e., vasopressin V2 receptor antagonism by tolvaptan, using, for example, AR-C124910XX or AR-C133913XX, has specific and clear advantages. For instance, administration of tolvaptan which blocks the vasopressin V2 receptor and thus causes severe polyuria (loss of water in the urine), nocturia (frequent urination in the night), thirst and dry mouth in a vast majority of the patients. Other common side effects of tolvaptan are constipation, loss of appetite, dry skin, nausea, vomiting, and potential for acute liver failure (Baur and Meaney, Pharmcother 34:605-616, 2014). Furthermore, tolvaptan is metabolized by the CYP3A4 system, which may result in increased interactions with other medications (Dixon and Lien, Ther Clin Risk Manag 4:1149-1155, 2008). In view of these untoward effects, the long-term use of tolvaptan for the treatment of ADPKD is not feasible in many patients. It should be noted that tolvaptan was originally developed for the treatment of hyponatremia, which represents a shorter period of treatment. It has also been shown that chronic blockade of vasopressin V2 receptor by tolvaptan results in increased endogenous production of AVP, which may have adverse effects on V1a and V1b receptors.

AR-C124910XX or M8 is a major metabolite of ticagrelor, and is formed by O-deethylation, and found mostly in plasma and feces. A second major metabolite of ticagrelor is AR-C133913XX or M5, found in urine and feces. AR-C124910XX and AR-C133913XX can be tested for their ability to decrease urinary AVP excretion by injecting them into the mice and determining the urinary AVP excretion. Thus, disclosed herein are composition comprising AR-C124910XX and/or AR-C133913XX or derivatives thereof and methods of decreasing AVP levels in subjects with a disease or condition associated with increased circulating levels of AVP.

Compositions

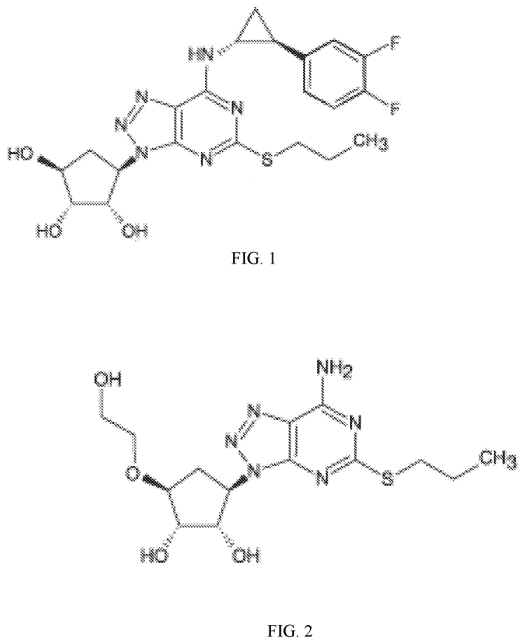

AR-C124910XX and AR-C133913XX, together or alone, can be useful for treating subjects with diseases or conditions associated with elevated AVP. FIGS. 1 and 2 show the structures of AR-C124910XX and AR-C133913XX, respectively.

In some aspects, the AR-C124910XX or AR-C133913XX can be used alone, in combination together or in combination with other therapeutic drugs used to treat subjects with diseases or conditions associated with elevated AVP. For example, in some aspects, AR-C124910XX or AR-C133913XX can be administered with tolvaptan. In some aspects, AR-C124910XX or AR-C133913XX can be administered with inhibitors of mTOR activity. In some aspects, the combination of AR-C124910XX or AR-C133913XX with tolvaptan can be used to potentiate their effects on inhibiting cyst growth or treat dilutional hyponatremia.

Methods of Treating Conditions or Diseases

Disclosed are methods for treating conditions or diseases associated with elevated arginine vasopressin (AVP) production or levels in a subject. In some aspects, the condition or disease can be a kidney disease. In some aspects, the condition or disease can be ADPKD. In some aspects, the condition or disease can be liver disease. In some aspects, the condition or disease can be condition or congestive heart failure. In some aspects, the condition or disease can be cirrhosis of the liver. In some aspect, the condition or disease can be cardiorenal syndrome. In some aspects, the condition or disease can be high altitude pulmonary hypertension. In some aspects, the condition or disease can be severe depression. In some aspects, the condition or disease can be the condition or disease can be autism. Also disclosed herein are methods for treating a kidney disease associated with elevated arginine vasopressin levels. In some aspects, the methods can comprise administering to the subject a composition comprising a therapeutically effective amount of AR-C124910XX or AR-C133913XX, thereby treating the condition or disease in the subject. In some aspects, the methods can comprise administering to the subject a composition comprising a therapeutically effective amount of AR-C124910XX or AR-C133913XX, thereby decreasing AVP production or levels in the subject.

In some aspects, treating liver disease comprises inhibiting AVP production in the subject. In some aspects, treating congestive heart failure comprises inhibiting AVP production in the subject. In some aspects, treating cirrhosis of the liver comprises inhibiting AVP production in the subject. In some aspects, treating high altitude pulmonary hypertension comprises inhibiting AVP production in the subject. In some aspects, treating severe depression comprises inhibiting AVP production in the subject. In some aspects, treating autism comprises inhibiting AVP production in the subject.

In some aspects, ADPKD can be associated with mutation of PKD1 gene and/or PKD2 gene. In some aspects, ADPKD can associated with altered expression of PKD1 gene and/or PKD2 gene. In some aspects, ADPKD can be associated with mutations of PKD1 gene and/or PKD2 gene.

Disclosed herein are methods of inhibiting arginine vasopressin production in a subject. In some aspects, the methods can comprise administering a composition comprising an effective amount of AR-C124910XX or AR-C133913XX to the subject, thereby inhibiting AVP production in the subject. Also disclosed herein are methods of lowering circulating levels arginine vasopressin production in a subject. In some aspects, the methods can comprise administering a composition comprising an effective amount of AR-C124910XX or AR-C133913XX to the subject, thereby lowering circulating AVP levels in the subject.

As used herein, the term “elevated” (such as elevated AVP) means a level higher than found in normal patients (e.g., patients without a kidney disease). In the case of elevated AVP, in some aspects, the determination of an elevated AVP can be based on comparison of measured plasma AVP levels of a subject to the main plasma AVP level and its standard deviation for a reference normal population. See for example, van Londen, L. et al. (1997) Neuropsychopharmacology 17(4):284-292 for reference values related to measurements for a normal control population (e.g., Table 2 on page 287) which is hereby incorporated by reference for its teaching of the same. In some aspects, elevated AVP can be based on comparison of measured urinary AVP level of a subject collected over a period (such as 24 hours) to the mean urinary AVP level and its standard deviation collected over the same duration for a reference normal population.

In some aspects, a subject can have an elevated AVP if the plasma AVP level of the subject is at least one standard deviation from the mean plasma AVP value (for example, determined in pg/ml; mean plasma AVP value plus one standard deviation) for a normal control population. In some aspects, the subject can have an elevated AVP if the plasma AVP level of the subject is greater than the mean plasma AVP value for a normal control population by at least 1.5 standard deviations. In some aspects, the subject can have an elevated AVP if the plasma AVP level of the subject is greater than the mean plasma AVP value for a normal control population by at least two standard deviations. In some aspects, the subject can have an elevated AVP if the plasma AVP level of the subject is greater than the mean plasma AVP value for a normal control population by at least 2.5 standard deviations. In some aspects, the subject can have an elevated AVP if the plasma AVP level of the subject is greater than the mean plasma AVP value for a normal control population by at least three standard deviations. In some aspects, the subject can have an elevated AVP if the plasma AVP level of the subject is greater than the mean plasma AVP value for a normal control population by at least 3.5 standard deviations. In some aspects, the subject can have an elevated AVP if the plasma AVP level of the subject is greater than the mean plasma AVP value for a normal control population by at least four standard deviations. In some aspects, the subject can have an elevated AVP if the plasma AVP level of the subject is greater than the mean plasma AVP value for a normal control population and is between 2.5 standard and 6 deviations from the mean plasma AVP value for a normal control population.

In some aspects, a subject can have an elevated AVP if the urinary AVP level of the subject is at least one standard deviation from the mean urinary AVP value (mean AVP value plus one standard deviation) for a normal control population. In some aspects, the subject can have an elevated AVP if the urinary AVP level of the subject is greater than the mean urinary AVP value for a normal control population by at least 1.5 standard deviations. In some aspects, the subject can have an elevated AVP if the urinary AVP level of the subject is greater than the mean urinary AVP value for a normal control population by at least two standard deviations. In some aspects, the subject can have an elevated AVP if the urinary AVP level of the subject is greater than the mean urinary AVP value for a normal control population by at least 2.5 standard deviations. In some aspects, the subject can have an elevated AVP if the urinary AVP level of the subject is greater than the mean urinary AVP value for a normal control population by at least three standard deviations. In some aspects, the subject can have an elevated AVP if the urinary AVP level of the subject is greater than the mean urinary AVP value for a normal control population by at least 3.5 standard deviations. In some aspects, the subject can have an elevated AVP if the urinary AVP level of the subject is greater than the mean urinary AVP value for a normal control population by at least four standard deviations. In some aspects, the subject can have an elevated AVP if the urinary AVP level of the subject is greater than the mean urinary AVP value for a normal control population and is between 2.5 standard and 6 deviations from the mean urinary AVP value for a normal control population.

In some aspects, ADPKD can be associated with reduced or altered activity of PKD1 protein and/or PKD2 protein. In some aspects, ADPKD can be associated with increased renal epithelial cell proliferation. In some aspects, ADPKD can be associated with bilateral renal enlargement and cyst.

In some aspects, treating kidney disease, liver disease, congestive heart failure, cirrhosis of the liver, cardiorenal syndrome, high altitude pulmonary hypertension, severe depression, and/or autism can comprise inhibiting AVP production in the subject. In some aspects, inhibiting AVP production can lower AVP plasma level in the subject. In some aspects, lowered AVP plasma level in the subject can be detected as a lower urinary AVP concentration or excretion by the subject.

In some aspects, treating any of the disease or conditions disclosed herein c a n comprise slowing down the progression of the disease or condition. In some aspects, treating kidney disease can comprise slowing down progression of the cystic disease. Slowing down the progression of the disease does not eliminate the disease but it can result in the subject not developing kidney failure and/or end-stage renal disease during their lifetime. In some aspects, slowing down progression of the disease or of the cystic disease can comprise reducing risk of developing kidney failure and/or end-stage renal disease. In some aspects, slowing down progression of the disease or of the cystic disease can comprise preventing an increase in the number of renal cysts, increase in size of renal cyst, and/or increase in size or mass of one or both kidneys. In some aspects, slowing down progression of the disease or of the cystic disease can comprise reducing number of renal cysts, reducing size of renal cyst, and/or reducing size or mass of one or both kidneys.

Disclosed herein are methods of inhibiting cyst growth in a kidney in a subject suffering from a kidney disease associated with elevated arginine vasopressin production or levels. In some aspects, the methods can comprise administering a composition comprising an effective amount of AR-C124910XX or AR-C133913XX to the subject, thereby inhibiting AVP production in the subject.

In some aspects, treating kidney disease can comprise inhibiting cAMP production in renal collecting duct cells of the subject. In some aspects, the renal collecting duct cell can be or can comprise a principal cell. In some aspects, treating kidney disease can increase urine output and/or decrease urine osmolarity. In some aspects, treating kidney disease can inhibit proliferation of renal epithelial cell.

In some aspects, treating a disease or a kidney disease associated with elevated AVP in a subject can comprise lowering the level of circulating AVP in the subject.

In some aspects, the subject can be a mammal. In some aspects, the mammal can a human.

In some aspects, the subject has been diagnosed with a need for treatment of elevated AVP production or levels prior to the administering step. The subject may be in need of treatment of a disease or condition associated with elevated AVP production or levels. In some aspects, the disease or condition can be kidney disease, liver disease, congestive heart failure, cirrhosis of the liver, cardiorenal syndrome, high altitude pulmonary hypertension, severe depression or autism. In some aspects, the disease or condition can be a kidney disease, a liver disease, congestive heart failure, cardiorenal syndrome, cirrhosis of liver, high altitude pulmonary hypertension, autism, autosomal dominant polycystic kidney disease (ADPKD), autosomal dominant polycystic liver disease or depression. Thus, the disclosed methods can, in some instances, further comprise the step of identifying a subject in need of treatment of kidney disease, liver disease, congestive heart failure, cirrhosis of the liver, cardiorenal syndrome, high altitude pulmonary hypertension, severe depression or autism. In some aspects, the disclosed methods can, in some instances, further comprise the step of identifying a subject in need of treatment of a kidney disease, a liver disease, congestive heart failure, cardiorenal syndrome, cirrhosis of liver, high altitude pulmonary hypertension, autism, autosomal dominant polycystic kidney disease (ADPKD), autosomal dominant polycystic liver disease or depression. Identifying a subject in need of treatment of kidney disease, liver disease, congestive heart failure, cirrhosis of the liver, cardiorenal syndrome, high altitude pulmonary hypertension, severe depression or autism can comprise ultrasound, CT or MM scans to check for kidney abnormality or blood tests to analyze known genetic defects related to ADPKD or elevated AVP production or levels.

Disclosed are methods for treating autosomal dominant polycystic kidney disease (ADPKD) in a subject. In some aspects, the methods can comprise administering to the subject a composition comprising a therapeutically effective amount of AR-C124910XX or AR-C133913XX, thereby treating ADPKD. In some aspects, treating ADPKD can comprise reducing cyst number and/or size or decreasing or preventing the increase of kidney size. Other symptoms of ADPKD can be affected during treatment with the disclosed compositions. Other treatable symptoms include, but are not limited to, size of abdomen, presence of kidney stones, high blood pressure, blood in the urine, urinary tract infections, gradual decrease in kidney function, and back and neck pain.

In some aspects, the subject does not have a coagulation disorder. In some aspects, the coagulation disorder can be a hypercoagulation disorder or thrombophilia. In some aspects, the hypercoagulation disorder or thrombophilia can be inherited hypercoagulable condition. In some aspects, the inherited hypercoagulable condition can be associated with and may include any of factor V Leiden mutation, prothrombin gene mutation, antithrombin III deficiency, protein C deficiency, protein S deficiency, elevated homocysteine level, elevated fibrinogen level, dysfibrinogenemia, elevated factor VIII level, factor XIII mutation, elevated factor IX level, elevated factor XI level, fibrinolysis disorder, plasminogen deficiency, and elevated plasminogen activator inhibitor (PAI-1).

In some aspects, coagulation disorder can be a bleeding disorder. In some aspects, the bleeding disorder can be congenital. The congenital bleeding disorder can include any of hemophilia, factor II deficiency, factor V deficiency, factor VII deficiency, factor X deficiency, factor XI deficiency, factor XII deficiency, factor XIII deficiency, von Willebrand's disease, Bernard-Soulier syndrome, complete plasminogen activator inhibitor 1 (PAI-1) deficiency, congenital afrinogenemia, glycoprotein VI deficiency, gray platelet syndrome, Noonan syndrome, prekallikrein deficiency, prothrombin deficiency, Stormorken syndrome, thrombocytopenia-ab sent radius (TAR) syndrome and Wiskott-Aldrich syndrome.

In some aspects, a diagnosis of ADPKD can comprise the presence of one or more of large echogenic kidneys without distinct macroscopic cysts at 50% risk for ADPKD, presence of bilateral renal enlargement and cysts, PKD1 gene mutation, PKD2 gene mutation, and mutation in modifiers of PKD1 expression or PKD2 expression.

In some aspects, treating any of the conditions or diseases disclosed herein can comprise reducing cyst number or size or decreasing kidney size. In some aspects, the method can further comprise ameliorating one or more symptoms associated with elevated AVP production or levels. In some aspects, one or more symptoms associated with elevated AVP production or levels including by not limited to ADPKD can include any of acute loin pain, haematuria, ballotable kidneys, sub arachnoid hemorrhage (berry aneurysm), hypertension, associated liver cyst, uremia due to renal failure, anemia due to CKD, increase RBC or erythropoietin secretion.

In some aspects, the disclosed methods can further comprise administering one or more additional therapeutics. Thus, in some instances, disclosed herein are methods for treating any of the conditions or diseases disclosed herein in a subject, the method comprising administering to the subject a composition comprising a therapeutically effective amount of AR-C124910XX or AR-C133913XX and one or more additional therapeutics. In some aspects, the one or more additional therapeutic can be a mTOR inhibitor, including but not limited to, Sirolimus, Everolimus (RAD001), Temsirolimus (CCI-779), Ridaforolimus (AP23573, MK-8669), Deforolimus, Dactolisib, BGT226, SF1126, PKI-587, Sapanisertib (INK128), AZD8055 and AZD2014. In AR-C124910XX and AR-C133913XX, the mTOR inhibitor can be Sirolimus and Everolimus. In some aspects, the one or more additional therapeutic can a somatostatin analogue, including but not limited to, Octreotide, Pasireotide (SOM230), dopastatin BIM-23A387, dopastatin BIM-23A760, somatostatin octapeptide-doxorubicin RC-121, somatostatin octapeptide-doxorubicin RC-160, somatostatin octapeptide-2-pyrrolino-DOX conjugate AN-201, AN-238 (AN-201 linked to RC-121), JF-10-81, 90Y-DOTATOC, 177Lu DOTATATE [177Lu]DOTA-Tyr(3)-octreotate and Lanreotide. In some aspects, the somatostatin analogue can be any of Octreotide and Lanreotide. In some aspects, the one or more additional therapeutic can be a vasopressin V2 receptor antagonist, including but not limited to, OPC-31260, Lixivaptan, Mozavaptan, Satavaptan, Conivaptan (YM-087), SR-121463A, VPA-985 and Tolvaptan (OPC-41061). In some aspects, the vasopressin V2 receptor antagonist can be any of OPC-31260 and Tolvaptan. In some aspects, the one or more additional therapeutic can be an epidermal growth factor receptor (EGFR) inhibitor, including but not limited to, bosutinib, gefitinib, lapatinib, cetuximab, panitumumab, vandetanib, neratinib, necitumumab, osimertnib and erlotinib. In some aspects, the epidermal growth factor receptor (EGFR) inhibitor can be any of bosutinib, gefitinib and erlotinib. A combination of mTOR inhibitors, somatostatin analogues, vasopressin V2 receptor antagonists and EGFR inhibitors can also be used. In some aspects, the one or more additional therapeutic can be administered in the same composition or in a separate composition as AR-C124910XX or AR-C133913XX. In some aspects, the additional therapeutic can be administered simultaneously or consecutively with the AR-C124910XX or AR-C133913XX.

In some aspects, the one or more additional therapeutic agents can be administered concurrently or sequentially with AR-C124910XX or AR-C133913XX. In some aspects, the one or more additional therapeutic agents can be administered before, after or taken with a composition comprising AR-C124910XX or AR-C133913XX. In some aspects, the composition comprising AR-C124910XX or AR-C133913XX can be administered orally, intravenously, subcutaneously or intramuscularly, as an implant or patch, or via a needle or microneedles. In some aspects, the one or more additional therapeutic agents can administered orally, intravenously, subcutaneously or intramuscularly, or as an implant or patch, or via a needle or microneedles.

In some aspects, one or more additional therapeutic agents can administered by the same route as a composition comprising AR-C124910XX or AR-C133913XX. In some aspects, one or more additional therapeutic can be administered by a different route as a composition comprising AR-C124910XX or AR-C133913XX.

Disclosed are methods for treating any of the conditions or disease disclosed herein in a subject comprising administering to the subject a composition comprising a therapeutically effective amount of ticagrelor or a metabolite of ticagrelor or a derivative of ticagrelor, thereby treating ADPKD, wherein the step of administering to the subject a composition comprising an effective amount of AR-C124910XX or AR-C133913XX is a long-term treatment regimen. A long-term treatment regimen or chronic treatment regimen means a course of treatment that lasts longer than 1 month to a life-time depending on the subject. In some aspects, long-term treatment regimen or chronic treatment regimen in such a subject can be 1, 2, 3, 4, 5, 6, 7, 8, 9, 10, 11, 12 months or longer. In some aspects, along-term treatment regimen or chronic treatment regimen can be 1, 2, 3, 4, 5, 6, 7, 8, 9, 10, 15, 20, 25, 30 years or longer. In some aspects, the long-term treatment regimen can last a lifetime.

In some aspects, the step of administering to the subject a composition comprising an effective amount of AR-C124910XX or AR-C133913XX can be a long-term treatment regimen. In some aspects, the long-term treatment regimen can be at least 2 weeks. In some aspects, the long-term treatment regimen can be at least 1 month. In some aspects, the long-term treatment regimen can be at least 1 year. In some aspects, the long-term treatment regimen can be at least 5 years. In some aspects, the long-term treatment regimen can be a lifetime from a diagnosis of need to treat.

In some aspects, the long-term treatment can be continuous. In some aspects, the long-term treatment can be discontinuous, wherein the treatment can be interrupted by one or more period in which administration of the composition comprising an effective amount of AR-C124910XX and AR-C133913XX can be withheld.

In some aspects, withholding a composition comprising an effective amount of AR-C124910XX and AR-C133913XX can be for a sufficient amount of time or for a specified amount of time. In some aspects, withholding a composition comprising an effective amount of AR-C124910XX and AR-C133913XX can be for a sufficient amount of time in which withholding can inhibit or reverse uncontrolled bleeding from the wound or internal ulcer. In some aspects, withholding a composition comprising an effective amount of AR-C124910XX or AR-C133913XX for a specified amount of time can reduce risk of uncontrolled bleeding associated with a procedure or therapy. In some aspects, the procedure can be surgery or intervention radiology. In some aspects, the therapy can increase bleeding time or decrease clotting time so as to place the subject at risk for uncontrolled bleeding.

In some aspects, decrease in AVP production can be determined by comparing AVP levels detected in the blood or urine of the subject before administering AR-C124910XX or AR-C133913XX to AVP levels detected in the blood or urine of the subject after administering AR-C124910XX or AR-C133913XX. In some aspects, AVP levels detected in the blood or urine of the subject after administering AR-C124910XX or AR-C133913XX can be detected at least one week after administering AR-C124910XX or AR-C133913XX.

In some instances, the dose of AR-C124910XX or AR-C133913XX can be 50, 60, 70, 80, 90, 100, 110, 120, 130, 140, 150 mg twice per day. In some instances, the same daily dose can be administered in a once per day form. In some instances, AR-C124910XX or AR-C133913XX can be administered daily, weekly, or monthly.

Methods for Treating Disease or Condition Associated with Elevated AVP

Disclosed are methods for treating a disease or condition associated with elevated AVP in a subject suffering therefrom. In some aspects, the methods can comprise the administering to the subject a composition comprising a therapeutically effective amount of AR-C124910XX or AR-C133913XX, thereby treating the disease in the subject.

In some aspects, the disease or condition can include but is not limited to any of hypertension, preeclampsia, congestive heart failure, cardiorenal syndrome, cirrhosis of liver, diabetic ketoacidosis, post-traumatic stress disorder (PTSD), countering effect of loop diuretics, high altitude pulmonary edema, autism, syndrome of inappropriate antidiuretic hormone (SIADH), autosomal dominant polycystic kidney disease (ADPKD), kidney disease, liver disease, high altitude pulmonary hypertension, severe depression, dilutional hyponatremia and disease associated with elevated activity of AVP-V2 receptor-cAMP axis. In some aspects, autism can be autism spectrum disorder (ASD).

In some aspects, elevated activity of the AVP-V2 receptor-cAMP axis can comprise elevated circulating AVP and/or elevated V2 receptor signaling in the subject. In some aspects, elevated V2 receptor signaling can comprise elevated cAMP level in a V2 receptor positive cell of the subject.

In some aspects, a V2 receptor positive cell can a renal collecting duct cell. In some aspects, the renal collecting duct cell can comprise a principal cell. In some aspects, the renal collecting duct cell can be a principal cell. In some aspects, the renal collecting duct cell can a renal epithelial cell. In some aspects, the renal epithelial cell can be a principal cell of the collecting duct. In some aspects, the principal cell can translocate aquaporin protein to apical plasma membrane. In some aspects, the aquaporin protein can be in a subapical vesicle prior to transport to apical plasma membrane. In some aspects, the principal cell can increase expression of the aquaporin gene. In some aspects, aquaporin can be aquaporin protein 2 (AQP2) and aquaporin protein 3 (AQP3).

In some aspects, translocation of aquaporin and/or increased expression of aquaporin gene can alter transepithelial water transport. In some aspects, alteration of transepithelial water transport can comprise increased re-absorption of water by the principal cell or renal collecting duct cell, decreased urine output, increased urine osmolarity, and/or increased urinary AVP excretion. In some aspects, increased urinary AVP excretion can positively correlate with an increased plasma AVP level.

In some aspects, treating a disease or a condition disclosed herein or associated with elevated AVP in the subject can comprise inhibiting AVP production in the hypothalamus. In some aspects, treating a disease or a condition disclosed herein or associated with elevated AVP in a subject can comprise lowering circulating level of AVP in the subject. In some aspects, lowering circulating level of AVP can reduce signaling by AVP-dependent V2 receptor in a cell of the subject. In some aspects, reducing signaling by AVP-dependent V2 receptor can decrease cAMP levels in the cell of the subject. In some aspects, lowering circulating level of AVP can reduce signaling by AVP-dependent V1 receptor in a cell of the subject. In some aspects, V1a receptor can be a V1a receptor or V1b receptor. In some aspects, reducing signaling by AVP-dependent V1 or V2 receptor can slow or reverse a disease or a condition associated with elevated AVP in the subject.

In some aspects, the cell of the subject can be a renal collecting duct cell. In some aspects, the renal collecting duct cell can be a renal epithelial cell. In some aspects, the renal collecting duct cell can be or can comprise a principal cell.

In some aspects, the principal cell can have a lower cAMP level. In some aspects, the renal collecting duct cell can have a lower cAMP level. In some aspects, the renal epithelial cell can have a lower cAMP level. Such a lower cAMP can be due to lower plasma level of AVP, such that second messenger signaling through production of cAMP by AVP-dependent receptor can be decreased. In some aspects, AVP-dependent receptor in principal cell, renal epithelial cell or renal collecting duct cell can be a V2 receptor.

In some aspects, lowering of cAMP level can result in a decrease number of aquaporin proteins on apical surface of the principal cell. In some aspects, lowering of cAMP levels can result in decreased expression of one or more aquaporin genes. In some aspects, aquaporin proteins can be any of aquaporin protein 2 (AQP2) or aquaporin protein 3 (AQP3). In some aspects, aquaporin protein can be aquaporin protein 2 (AQP2).

In some aspects, decreased number of aquaporin proteins on apical surface of the principal cell can result in a decreased re-absorption of water by the principal cell or renal collecting duct cell, increased urine output, decreased urine osmolarity, and/or decreased urinary AVP excretion. In some aspects, decreased urinary AVP excretion can positively correlate with a decreased plasma AVP level.

In some aspects, treating a disease, a condition, or a kidney or liver disease associated with elevated AVP in a subject can comprise lowering level of circulating AVP in the subject. In some aspects, the subject may be in need of treatment of a kidney disease or liver disease. In some aspects, the subject may be in need of treatment for congestive heart failure, cirrhosis of the liver, cardiorenal syndrome, high altitude pulmonary hypertension, severe depression or autism.

In some aspects, the subject can free of or does not have a coagulation disorder. In some aspects, the coagulation disorder can be a hypercoagulation disorder or thrombophilia. In some aspects, the hypercoagulation disorder or thrombophilia can be inherited hypercoagulable condition. In some aspects, the inherited hypercoagulable condition can be associated with and can include but is not limited to any of factor V Leiden mutation, prothrombin gene mutation, antithrombin III deficiency, protein C deficiency, protein S deficiency, elevated homocysteine level, elevated fibrinogen level, dysfibrinogenemia, elevated factor VIII level, factor XIII mutation, elevated factor IX level, elevated factor XI level, fibrinolysis disorder, plasminogen deficiency and an elevated plasminogen activator inhibitor (PAI-1).

In some aspects, the coagulation disorder can be a bleeding disorder. In some aspects, the bleeding disorder can be congenital. The congenital bleeding disorder can be include but is not limited to any of hemophilia, factor II deficiency, factor V deficiency, factor VII deficiency, factor X deficiency, factor XI deficiency, factor XII deficiency, factor XIII deficiency, von Willebrand's disease, Bernard-Soulier syndrome, plasminogen activator inhibitor 1 (PAI-1) deficiency, congenital afrinogenemia, glycoprotein VI deficiency, gray platelet syndrome, Noonan syndrome, prekallikrein deficiency, prothrombin deficiency, Stormorken syndrome, thrombocytopenia-absent radius (TAR) syndrome and Wiskott-Aldrich syndrome.

In some aspects, the methods disclosed herein can further comprise ameliorating one or more symptoms associated with ADPKD. In some aspects, one or more symptoms associated with ADPKD can include but is not limited to any of acute loin pain, haematuria, ballotable kidneys, sub arachnoid hemorrhage (berry aneurysm), hypertension, associated liver cyst, uremia due to renal failure, anemia due to CKD, increase RBC or erythropoietin secretion.

In some aspects, the methods can further comprise administering one or more additional therapeutic agents. In some aspects, the one or more additional therapeutic agents can be an mTOR inhibitor. In some aspects, the mTOR inhibitor may include but is not limited to any of Sirolimus, Everolimus (RAD001), Temsirolimus (CCI-779), Ridaforolimus (AP23573, MK-8669), Deforolimus, Dactolisib, BGT226, SF1126, PKI-587, Sapanisertib (INK128), AZD8055 and AZD2014. In some aspects, the mTOR inhibitor can be Sirolimus or Everolimus (RAD001).

In some aspects, the one or more additional therapeutic agents can be a somatostatin analogue. In some aspects, the somatostatin analogue can include but is not limited to any of Octreotide, Pasireotide (SOM230), dopastatin BIM-23A387, dopastatin BIM-23A760, somatostatin octapeptide-doxorubicin RC-121, somatostatin octapeptide-doxorubicin RC-160, somatostatin octapeptide-2-pyrrolino-DOX conjugate AN-201, AN-238 (AN-201 linked to RC-121), JF-10-81, 90Y-DOTATOC, 177Lu DOTATATE [177Lu]DOTA-Tyr(3)-octreotate and Lanreotide. In some aspects, the somatostatin analogue can be Octreotide or Lanreotide.

In some aspects, the one or more additional therapeutic agents can be a vasopressin V2 receptor antagonist. In some aspects, the vasopressin V2 receptor antagonist can include but is not limited to any of OPC-31260, Lixivaptan, Mozavaptan, Satavaptan, Conivaptan (YM-087), SR-121463A, VPA-985 and Tolvaptan (OPC-41061). In some aspects, the vasopressin V2 receptor antagonist can be OPC-31260 and Tolvaptan.

In some aspects, the one or more additional therapeutic agents can be an epidermal growth factor receptor inhibitor. In some aspects, the epidermal growth factor receptor inhibitor can include but is not limited to bosutinib, gefitinib, lapatinib, cetuximab, panitumumab, vandetanib, neratinib, necitumumab, osimertnib, and erlotinib. In some aspects, the epidermal growth factor receptor inhibitor can be bosutinib, gefitinib, or erlotinib.

In some aspects, the one or more additional therapeutic agents can be administered concurrently or sequentially with a metabolite of ticagrelor. In some aspects, the metabolite of ticagrelor can be AR-C124910XX or AR-C133913XX. In some aspect, the one or more additional therapeutic agents can be administered before, after or taken with a composition comprising a ticagrelor metabolite. In some aspects, the composition comprising a ticagrelor metabolite can be administered orally, intravenously, subcutaneously or intramuscularly, as an implant or patch, or via a needle or microneedles. In some aspects, the one or more additional therapeutic agents can be administered orally, intravenously, subcutaneously or intramuscularly, or as an implant or patch, or via a needle or microneedles.

In some aspects, one or more additional therapeutic agents can be administered by the same route as a composition comprising a ticagrelor metabolite. In some aspects, one or more additional therapeutic agents can be administered by a different route as a composition comprising the ticagrelor metabolite.

In some aspects, the step of administering to the subject a composition comprising an effective amount of a ticagrelor metabolite can be a long-term treatment regimen. In some aspects, the long-term treatment regimen can be at least 2 weeks. In some aspects, the long-term treatment regimen can be at least 1 month. In some aspects, the long-term treatment regimen can be at least 1 year. In some aspects, the long-term treatment regimen can be at least 5 years. In some aspects, the long-term treatment regimen can be a lifetime from a diagnosis of need to treat.

In some aspects, the long-term treatment can be continuous. In some aspects, the long-term treatment can be discontinuous, wherein the treatment can be interrupted by one or more period in which administration of the composition comprising an effective amount of a ticagrelor metabolite can be withheld.

In some aspects, withholding a composition comprising an effective amount of a ticagrelor metabolite can be for a sufficient amount of time or for a specified amount of time. In some aspects, withholding a composition comprising an effective amount of a ticagrelor metabolite can be for a sufficient amount of time in which withholding inhibits or reverses uncontrolled bleeding from the wound or internal ulcer. In some aspects, withholding a composition comprising an effective amount of a ticagrelor metabolite for a specified amount of time can reduce risk of uncontrolled bleeding associated with a procedure or therapy. In some aspects, the procedure can be surgery or intervention radiology. In some aspects, the therapy can increase bleeding time or decrease clotting time so as to place the subject at risk for uncontrolled bleeding.

In some aspects, decrease in AVP production can be determined by comparing AVP levels detected in the blood or urine of the subject before administering a ticagrelor metabolite to AVP levels detected in the blood or urine of the subject after administering a ticagrelor metabolite. In some aspects, AVP levels detected in the blood or urine of the subject after administering a ticagrelor metabolite can be detected at least one week after administering a ticagrelor metabolite.

In some instances, the dose of a tricagrelor metabolite can be 50, 60, 70, 80, 90, 100, 110, 120, 130, 140, 150 mg twice per day. In some instances, the same daily dose can received in a once per day form. In some instances, a tricagrelor metabolite can be taken daily, weekly, or monthly.

Methods of Decreasing Arginine Vasopressin (AVP)

Disclosed are methods of decreasing arginine vasopressin (AVP) production in a subject. In some aspects, the methods can comprise administering to the subject a composition comprising an effective amount of AR-C124910XX or AR-C133913XX, thereby decreasing AVP production. In some aspects, the decrease in AVP production can be determined by comparing AVP levels detected in the blood or urine of the subject before administering the ticagrelor metabolite to AVP levels detected in the blood or urine of the subject after administering the ticagrelor metabolite. AVP levels in the urine can correlate to the levels in the plasma, therefore using urine or blood samples for detection of AVP levels can be appropriate.

In some aspects, AVP levels detected in the blood or urine of the subject after administering daily doses of AR-C124910XX or AR-C133913XX can be detected at least one week after administering AR-C124910XX or AR-C133913XX. In some aspects, AVP levels can be detected, 1, 2, 3, 4, 5, 6, 7, 8, 9, 10, 11, 12, 13, or 14 days after administering AR-C124910XX or AR-C133913XX. In some aspects, AVP levels can be detected 2, 4, 6, 7, 8, 10, or 12 weeks after administering daily doses of AR-C124910XX or AR-C133913XX.

In some aspects, the method can include any of the other methods disclosed herein.

Methods for Treating Dilutional Hyponatremia

Hyponatremia is a disorder characterized by an excess of body water relative to body content of sodium. Hyponatremia is the most common electrolyte disorder encountered in hospitalized patients (25-30%), associated with increased morbidity and mortality. Hyponatremia also represents a huge economic burden on the health care system, typically costing $10,000 more per hospitalized patient, over other hospitalized patients that do not have hyponatremia. It is often seen in patients with congestive heart failure (CHF), cirrhosis of liver and in SIADH (syndrome of inappropriate anti-diuretic hormone secretion) among others. In these conditions, the plasma levels of AVP are inappropriately high.

Disclosed are methods for treating dilutional hyponatremia in a subject comprising the step of administering to the subject a composition comprising an effective amount of a ticagrelor metabolite, thereby decreasing AVP production. In some aspects, the ticagrelor metabolite can be AR-C124910XX or AR-C133913XX.

Disclosed are methods for treating dilutional hyponatremia in a subject comprising the step of administering to the subject a composition comprising an effective amount of a ticagrelor metabolite, thereby decreasing AVP production, further comprising ameliorating one or more symptoms associated with dilutional hyponatremia. In some aspects, the ticagrelor metabolite can be AR-C124910XX or AR-C133913XX. In some aspects, one or more symptom associated with dilutional hyponatremia can be, but not limited to, fatigue, headache, muscle spasms, muscle cramps, confusion or hallucination.

Disclosed are methods for treating dilutional hyponatremia in a subject comprising the step of administering to the subject a composition comprising an effective amount of a ticagrelor metabolite, thereby decreasing AVP production, further comprising administering an additional therapeutic agent. In some aspects, the ticagrelor metabolite can be AR-C124910XX or AR-C133913XX. In some aspects, the additional therapeutic can be tolvaptan, a known vasopressin V2 receptor antagonist for treating hyponatremia. In some aspects, the additional therapeutic agent can be administered in the same composition or in a separate composition. In some aspects, the additional therapeutic agent can be administered simultaneously or consecutively with the ticagrelor metabolite.

In some aspects, the method can further comprise ameliorating one or more symptoms associated with dilutional hyponatremia. The method can further comprise administering an additional therapeutic agent, in addition to a composition comprising an effective amount of a ticagrelor metabolite. In some aspects, the additional therapeutic agent can be a vasopressin V2 receptor antagonist. In some aspects, vasopressin V2 receptor antagonist can include but is not limited to OPC-31260, Lixivaptan, Mozavaptan, Satavaptan, Conivaptan (YM-087), SR-121463A, VPA-985 and Tolvaptan (OPC-41061). In some aspects, the vasopressin V2 receptor antagonist can tolvaptan.

In some aspects, the subject can be a mammal. In some aspects, the mammal can a human, non-human primate, rabbit, sheep, rat, dog, cat, pig, or mouse. Non-human primates include but are not limited to monkeys, chimpanzees, gorillas, apes, lemurs, macaques and gibbons. In some aspects, the non-human primate can be a monkey or a chimpanzee.

Methods of Reducing One or More Symptoms Associated with ADPKD

Disclosed are methods of reducing one or more symptoms associated with ADPKD in a subject, comprising the step of administering to the subject a composition comprising an effective amount of a ticagrelor metabolite, so as to reduce circulating levels of AVP, thereby reducing one or more symptoms associated with ADPKD in the subject. In some aspects, the ticagrelor metabolite can be AR-C124910XX or AR-C133913XX.

In some aspects, one or more symptoms associated with ADPKD can be acute loin pain, haematuria, ballotable kidneys, subarachnoid hemorrhage (berry aneurysm), hypertension, associated liver cyst, uremia due to renal failure, anemia due to CKD, increase RBC or erythropoietin secretion.

In some aspects, the method can further comprise administering one or more additional therapeutic agents. The additional therapeutic agent can be a mTOR inhibitor, a somatostatin analogue, a vasopressin V2 receptor antagonist and an epidermal growth factor receptor inhibitor. Examples of said therapeutic agents are provided elsewhere in the application. In some aspects, the additional therapeutic agent can be Sirolimus, Everolimus (RAD001), Temsirolimus (CCI-779), Ridaforolimus (AP23573, MK-8669), Deforolimus, Dactolisib, BGT226, SF1126, PKI-587, Sapanisertib (INK128), AZD8055, AZD2014, Octreotide, Pasireotide (SOM230), dopastatin BIM-23A387, dopastatin BIM-23A760, somatostatin octapeptide-doxorubicin RC-121, somatostatin octapeptide-doxorubicin RC-160, somatostatin octapeptide-2-pyrrolino-DOX conjugate AN-201, AN-238 (AN-201 linked to RC-121), JF-10-81, 90Y-DOTATOC, 177Lu DOTATATE [177Lu]DOTA-Tyr(3)-octreotate, Lanreotide, OPC-31260, Lixivaptan, Mozavaptan, Satavaptan, Conivaptan (YM-087), SR-121463A, VPA-985 and Tolvaptan (OPC-41061), bosutinib, gefitinib, lapatinib, cetuximab, panitumumab, vandetanib, neratinib, necitumumab, osimertnib and erlotinib. In some aspects, the additional therapeutic agent can be Sirolimus, Everolimus (RAD001), Octreotide, Lanreotide, OPC-31260, Tolvaptan, bosutinib, gefitinib or erlotinib. In some aspects, the additional therapeutic agent can be tolvaptan.

In some aspects, the subject can be a mammal. The mammal can be a human, non-human primate, rabbit, sheep, rat, dog, cat, pig, or mouse. Examples of non-human primates include but are not limited to monkeys, chimpanzees, gorillas, apes, lemurs, macaques and gibbons. In some aspects, the non-human primate can be a monkey or a chimpanzee. In some aspects, the subject can be a human. In some aspects, the subject can a human in need of a treatment. In some aspects, the subject can a human in need of a treatment for one or more symptom associated with ADPKD or elevated plasma AVP level.

Methods for Treating ADPKD

Disclosed are methods for treating ADPKD in a subject suffering therefrom, comprising the step of administering to the subject a composition comprising a therapeutically effective amount of a ticagrelor metabolite, thereby treating ADPKD in the subject. In some aspects, the ticagrelor metabolite can be AR-C124910XX or AR-C133913XX.

In some aspects, the subject does not have an elevated level of plasma AVP and/or elevated level of urinary AVP. In some aspects, the subject can be free of an elevated level of plasma AVP and/or elevated level of urinary AVP. In some aspects, the subject can be a human, a non-human primate, a rabbit, a sheep, a rat, a dog, a cat, a pig, or a mouse.

In some aspects, administering to the subject a composition comprising a therapeutically effective amount of a ticagrelor metabolite can comprise reducing cyst number and/or size or decreasing or preventing the increase of kidney size.

In some aspects, administering to the subject a composition comprising a therapeutically effective amount of a ticagrelor metabolite can alleviate or reduce one or more symptoms associated with ADPKD. In some aspects, the one or more symptoms associated with ADPKD cab include but is not limited to acute loin pain, haematuria, ballotable kidneys, sub arachnoid hemorrhage (berry aneurysm), hypertension, associated liver cyst, uremia due to renal failure, anemia due to CKD, increase RBC or erythropoietin secretion. In some aspects, the one or more symptoms associated with ADPKD can include but is not limited to size of abdomen, presence of kidney stones, high blood pressure, blood in the urine, urinary tract infections, gradual decrease in kidney function, and back and neck pain.

In some aspects, the method can further comprise administering one or more additional therapeutic agents. The additional therapeutic agent can be a mTOR inhibitor, a somatostatin analogue, a vasopressin V2 receptor antagonist and an epidermal growth factor receptor inhibitor. Said inhibitors are described elsewhere in the application.

In some aspects, the additional therapeutic agent can be Sirolimus, Everolimus (RAD001), Temsirolimus (CCI-779), Ridaforolimus (AP23573, MK-8669), Deforolimus, Dactolisib, BGT226, SF1126, PKI-587, Sapanisertib (INK128), AZD8055, AZD2014, Octreotide, Pasireotide (SOM230), dopastatin BIM-23A387, dopastatin BIM-23A760, somatostatin octapeptide-doxorubicin RC-121, somatostatin octapeptide-doxorubicin RC-160, somatostatin octapeptide-2-pyrrolino-DOX conjugate AN-201, AN-238 (AN-201 linked to RC-121), JF-10-81, 90Y-DOTATOC, 177Lu DOTATATE [177Lu]DOTA-Tyr(3)-octreotate, Lanreotide, OPC-31260, Lixivaptan, Mozavaptan, Satavapta, Conivaptan (YM-087), SR-121463A, VPA-985 and Tolvaptan (OPC-41061), bosutinib, gefitinib, lapatinib, cetuximab, panitumumab, vandetanib, neratinib, necitumumab, osimertnib and erlotinib. In some aspects, the additional therapeutic can be Sirolimus, Everolimus (RAD001), Octreotide, Lanreotide, OPC-31260, Tolvaptan, bosutinib, gefitinib or erlotinib. In some aspects, the additional therapeutic agent can be tolvaptan.

In some aspects, treating ADPKD in the subject with a composition comprising a therapeutically effective amount of a ticagrelor metabolite, can be a long-term treatment regimen or chronic treatment regimen. In some aspects, the ticagrelor metabolite can be AR-C124910XX or AR-C133913XX.

In some aspects, the long-term treatment regimen can be at least 2 weeks. In some aspects, the long-term treatment regimen can be at least 1 month. In some aspects, the long-term treatment regimen can be at least 1 year. In some aspects, the long-term treatment regimen can be at least 5 years. In some aspects, the long-term treatment regimen can be a lifetime from a diagnosis of need to treat.

In some aspects, the long-term treatment can be continuous. In some aspects, the long-term treatment can be discontinuous, wherein the treatment can be interrupted by one or more periods in which administration of the composition comprising an effective amount of a ticagrelor metabolite can be withheld. In some aspects, the ticagrelor metabolite can be AR-C124910XX or AR-C133913XX.

In some aspects, withholding a composition comprising an effective amount of a ticagrelor metabolite can be for a sufficient amount of time or for a specified amount of time. In some aspects, ADPKD in a subject suffering therefrom can be treated with a composition comprising a therapeutically effective amount of a ticagrelor metabolite irrespective of coagulation status. In some aspects, the ticagrelor metabolite can be AR-C124910XX or AR-C133913XX.

In some aspects, ADPKD in a subject suffering therefrom can be treated with a composition comprising a therapeutically effective amount of a ticagrelor metabolite taking into consideration coagulation status. In some aspects, the ticagrelor metabolite can be AR-C124910XX or AR-C133913XX.

In some aspects, the subject can be free of a coagulation disorder. In some aspects, the coagulation disorder can be a hypercoagulation disorder or thrombophilia. In some aspects, the hypercoagulation disorder or thrombophilia can be inherited hypercoagulable condition. In some aspects, the inherited hypercoagulable condition can be associated with and can include any of factor V Leiden mutation, prothrombin gene mutation, antithrombin III deficiency, protein C deficiency, protein S deficiency, elevated homocysteine level, elevated fibrinogen level, dysfibrinogenemia, elevated factor VIII level, factor XIII mutation, elevated factor IX level, elevated factor XI level, fibrinolysis disorder, plasminogen deficiency and elevated plasminogen activator inhibitor (PAI-1).

In some aspects, the coagulation disorder can be a bleeding disorder. In some aspects, the bleeding disorder can be congenital. The congenital bleeding disorder can be hemophilia, factor II deficiency, factor V deficiency, factor VII deficiency, factor X deficiency, factor XI deficiency, factor XII deficiency, factor XIII deficiency, von Willebrand's disease, Bernard-Soulier syndrome, complete plasminogen activator inhibitor 1 (PAI-1) deficiency, congenital afrinogenemia, glycoprotein VI deficiency, gray platelet syndrome, Noonan syndrome, prekallikrein deficiency, prothrombin deficiency, Stormorken syndrome, thrombocytopenia-absent radius (TAR) syndrome or Wiskott-Aldrich syndrome.

In some aspects, the subject can have a hypercoagulation disorder or thrombophilia and treatment with a tricagrelor metabolite can treat both ADPKD and its symptoms as well as reduce or alleviate hypercoagulation disorder or thrombophilia. In some aspects, the ticagrelor metabolite can be AR-C124910XX or AR-C133913XX. In some aspects, the hypercoagulation disorder or thrombophilia can be inherited hypercoagulable condition. In some aspects, the inherited hypercoagulable condition can be associated with and can include any of factor V Leiden mutation, prothrombin gene mutation, antithrombin III deficiency, protein C deficiency, protein S deficiency, elevated homocysteine level, elevated fibrinogen level, dysfibrinogenemia, elevated factor VIII level, factor XIII mutation, elevated factor IX level, elevated factor XI level, fibrinolysis disorder, plasminogen deficiency and elevated plasminogen activator inhibitor (PAI-1).

In some aspects, the dose of the tricagrelor metabolite can be 50, 60, 70, 80, 90, 100, 110, 120, 130, 140, 150 mg twice per day. In some instances, the same daily dose can be administered in a once per day form. In some aspects, the tricagrelor metabolite can be administered daily, weekly, or monthly. In some aspects, the ticagrelor metabolite can be AR-C124910XX or AR-C133913XX.

Methods of Decreasing Proliferation of Medullary Collecting Duct Cells

Disclosed are methods of decreasing proliferation of medullary collecting duct cells comprising administering to a subject a composition comprising an effective amount of a ticagrelor metabolite, thereby decreasing proliferation of medullary collecting duct cells. In some aspects, the ticagrelor metabolite can be AR-C124910XX or AR-C133913XX. In some aspects, increasing concentrations of a ticagrelor metabolite can cause a decrease in the proliferation of medullary collecting duct cells.

Compositions and Administration

In some aspects, the disclosed compositions can be pharmaceutical compositions comprising ticagrelor metabolites. In some aspects, the ticagrelor metabolite can be AR-C124910XX. In some aspects, the ticagrelor metabolite can be AR-C133913XX. In some aspects, the pharmaceutical composition can comprise a therapeutically effective amount of a ticagrelor metabolite. In some aspects, the pharmaceutical composition can further comprise a pharmaceutically acceptable carrier.

In some aspects, the pharmaceutical carrier can be, for example, a solid, liquid, or gas. Examples of solid carriers include lactose, terra alba, sucrose, talc, gelatin, agar, pectin, acacia, magnesium stearate, and stearic acid. Examples of liquid carriers include but are not limited to sugar syrup, peanut oil, olive oil, and water. Examples of gaseous carriers include but are not limited to carbon dioxide and nitrogen.

In some aspects, administration or delivery of any of the therapeutic agents disclosed herein to a subject can be via a variety of mechanisms. For example, the therapeutic agent can be formulated as a pharmaceutical composition.

Pharmaceutical compositions can be administered in a number of ways depending on whether local or systemic treatment is desired, and on the area to be treated.

Preparations of parenteral administration include sterile aqueous or non-aqueous solutions, suspensions, and emulsions. Examples of non-aqueous solvents include but are not limited to propylene glycol, polyethylene glycol, vegetable oils such as olive oil, and injectable organic esters such as ethyl oleate. Aqueous carriers include but are not limited to water, alcoholic/aqueous solutions, emulsions or suspensions, including saline and buffered media. Parenteral vehicles include but are not limited to sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like. Formulations for optical administration can include but are not limited to ointments, lotions, creams, gels, drops, suppositories, sprays, liquids and powders. Conventional pharmaceutical carriers, aqueous, powder or oily bases, thickeners and the like may be necessary or desirable. Compositions for oral administration can include powders or granules, suspensions or solutions in water or non-aqueous media, capsules, sachets, or tablets. Thickeners, flavorings, diluents, emulsifiers, dispersing aids, or binders may be desirable. Some of the compositions can be administered as a pharmaceutically acceptable acid- or base-addition salt, formed by reaction with inorganic acids such as hydrochloric acid, hydrobromic acid, perchloric acid, nitric acid, thiocyanic acid, sulfuric acid, and phosphoric acid, and organic acids such as formic acid, acetic acid, propionic acid, glycolic acid, lactic acid, pyruvic acid, oxalic acid, malonic acid, succinic acid, maleic acid, and fumaric acid, or by reaction with an inorganic base such as sodium hydroxide, ammonium hydroxide, potassium hydroxide, and organic bases such as mono-, di-, trialkyl and aryl amines and substituted ethanolamines.

Kits

The compositions and therapeutic agents described herein can be packaged together in any suitable combination as a kit useful for performing, or aiding in the performance of, any of the disclosed methods. In some aspects, the kit components can be designed and adapted for use together in a disclosed method. For example, a disclosed kit for treating diseases or conditions associated with elevated AVP can comprise a composition comprising AR-C124910XX or AR-C133913XX and instructions for how to administer the composition to a subject for treatment of diseases or conditions associated with elevated AVP.

### EXAMPLES

### Example 1: Ticagrelor Metabolites Decrease AVP Production

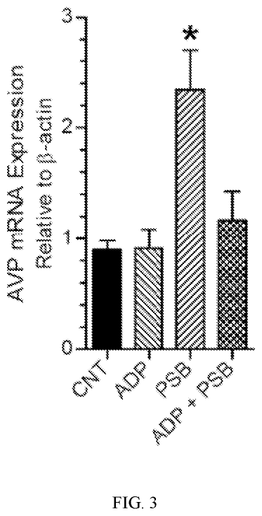

The blockade of P2Y12 receptor by PSB-0739 on AVP gene expression in cultured rat hypothalamic cells was studied. As shown in FIG. 3, blockade of P2Y12 receptor by PSB-0739 significantly increased the mRNA expression of the AVP gene. This result shows that the observed increase in AVP production following administration of clopidogrel to rats was due to direct blockade of P2Y12 receptor in hypothalamus by active metabolites of these drugs. Interestingly, the effect of PSB-0739 on AVP gene expression was blocked by ADP, the agonist of P2Y12 receptor.

The effect of prasugrel on urinary concentration ability in mice was also tested and the same findings as clopidogrel were observed. When administered to mice, prasugrel also caused significantly increased urine concentrating ability associated with significantly increased urinary excretion of AVP and protein abundance of AQP2 in the kidney medulla.

Both clopidogrel bisulfate and prasugrel belong to thienopyridine group of drugs, and thus both are prodrugs that need to be metabolized in the liver releasing their active metabolites that block the platelet P2Y12 receptor. In view of these results, the effects of ticagrelor, which is not a prodrug, on urinary concentrating ability and AVP production in mice was tested.

Few differences between the thienopyridine group of drugs and ticagrelor exist. The thienopyridine group are pro-drugs that are metabolized by the cytochrome system in the liver releasing active metabolites. It is the active metabolites that bind to P2Y12 receptor covalently. Thus, binding of the active metabolites of clopidogrel or prasugrel to P2Y12 receptors in platelets is irreversible and the binding remains through the lifespan of the platelets. This creates problems in patients taking a thienopyridine group of anti-thrombotic drugs that go through an emergency surgical procedure. Since the percentage of active metabolite formed is dependent on liver function, in patients with reduced liver function higher doses of these drugs are needed to compensate. In contrast, ticagrelor is not a prodrug and it directly binds to the P2Y12 receptor without the need for forming active metabolite. Ticagrelor also binds to P2Y12 receptor reversibly, thus allowing surgical procedures after 24-hour wash period.

The effect of ticagrelor on mice showed that instead of concentrating urine, the drug caused dilution of urine with increased urine flow associated with decreased urinary excretion of AVP. When tested, the effect of different doses of ticagrelor, it was found that the above effects were dose-dependent. However, when tested directly on primary cultures of rat inner medullary collecting duct cells, ticagrelor caused concentration-dependent increases in the mRNA expression in the dDAVP-induced AQP2 and AQP3 water channel proteins, did not interfere with the dDAVP-induced increase in cAMP production.

In sum, ticagrelor binds to P2Y12 receptor in medullary collecting duct cells and enhances the effect on dDAVP on the mRNA expression of AQP2 and AQP3 water channels and cAMP production. Thus, the observed decreased urinary concentration in ticagrelor-treated mice was not because of the effect of ticagrelor on renal medullary collecting duct.

The results suggest that decreased production of AVP in ticagrelor is causing decreased concentration of urine in ticagrelor-treated mice. The blockade of P2Y12 receptor by ticagrelor is not likely causing decreased production of AVP because the blockade of P2Y12 receptor by PSB-0739, a potent and selective antagonist of P2Y12 receptor, causes significant increase in the production of mRNA of AVP (FIG. 3).

Taken together, the decreased production of AVP in ticagrelor-treated mice may be due to binding to a site other than P2Y12 receptor in hypothalamic cells and causing a decrease in the mRNA expression of AVP and thus decreased production of AVP. This off-target effect appears to be dependent on the dose of ticagrelor.

Alternatively, the observed in vivo effect of ticagrelor in mice can be the due to a ticagrelor metabolite such as AR-C124910XX or AR-C133913XX, which may bind to hypothalamic cells at a site other than P2Y12 receptor and exert inhibitory effect on the production of AVP. Data showed that ticagrelor did not inhibit the baseline mRNA expression of AVP, but actually increased it by about a 1.8-fold. Based on this observation on cultured hypothalamic cells and the unequivocal data obtained from the primary cultures of rat inner medullary collecting duct cells, the observed in vivo effect of ticagrelor on decreased AVP production is mediated by the action of one or both metabolites of ticagrelor (AR-C124910XX or AR-C133913XX).

What is claimed is:

1. A method for inhibiting arginine vasopressin (AVP) production in a subject, the method comprising administering a composition comprising an effective amount of AR-C124910XX or AR-C133913XX to the subject, thereby inhibiting AVP production in the subject.

2. A method for treating a kidney disease associated with elevated arginine vasopressin (AVP) levels in a subject, the method comprising administering a composition comprising an effective amount of AR-C124910XX or AR-C133913XX to the subject, thereby decreasing AVP production in the subject.

3. A method for treating a liver disease associated with elevated arginine vasopressin (AVP) levels in a subject, the method comprising administering a composition comprising an effective amount of AR-C124910XX or AR-C133913XX to the subject, thereby decreasing AVP production in the subject.

4. A method for lowering circulating levels of arginine vasopressin (AVP) in a subject, the method comprising administering a composition comprising an effective amount of AR-C124910XX or AR-C133913XX to the subject, thereby lowering circulating levels of AVP in the subject.

5. A method of inhibiting cyst growth in a kidney in a subject suffering from a kidney disease associated with elevated arginine vasopressin (AVP) levels, the method comprising administering a composition comprising an effective amount of AR-C124910XX or AR-C133913XX to the subject, thereby inhibiting cyst grown in the kidney of the subject.

6. A method for treating congestive heart failure in a subject, the method comprising administering a composition comprising an effective amount of AR-C124910XX or AR-C133913XX to the subject, thereby decreasing AVP production in the subject.

7. A method for treating cirrhosis of the liver in a subject, the method comprising administering a composition comprising an effective amount of AR-C124910XX or AR-C133913XX to the subject, thereby decreasing AVP production in the subject.

8. A method for treating cardiorenal syndrome in a subject, the method comprising administering a composition comprising an effective amount of AR-C124910XX or AR-C133913XX to the subject, thereby decreasing AVP production in the subject.

9. A method for treating high altitude pulmonary hypertension in a subject, the method comprising administering a composition comprising an effective amount of AR-C124910XX or AR-C133913XX to the subject, thereby decreasing AVP production in the subject.

10. A method for treating severe depression in a subject, the method comprising administering a composition comprising an effective amount of AR-C124910XX or AR-C133913XX to the subject, thereby decreasing AVP production in the subject.

11. A method for treating autism in a subject, the method comprising administering a composition comprising an effective amount of AR-C124910XX or AR-C133913XX to the subject, thereby decreasing AVP production in the subject.

12. The method of claim 1, wherein the inhibiting AVP production lowers AVP plasma level in the subject.

13. The method of claim 12, wherein lowered AVP plasma level in the subject is detected as a lower urinary AVP concentration or excretion by the subject.

14. The method of claim 1, wherein the subject has a kidney disease, a liver disease, congestive heart failure, cardiorenal syndrome, cirrhosis of liver, high altitude pulmonary hypertension, autism, autosomal dominant polycystic kidney disease (ADPKD), autosomal dominant polycystic liver disease or depression.

15. The method of claim 2, wherein treating kidney disease comprises slowing down the progression of the disease.

16. The method of claim 2, wherein the kidney disease is an autosomal dominant polycystic kidney disease (ADPKD).

17. The method of claim 16, wherein the ADPKD is associated with a mutation of the PKD1 gene and/or the PDK2 gene.

18. The method of claim 16, wherein ADPKD is associated with altered expression of PKD1 gene and/or PKD2 gene.

19. The method of claim 18, wherein altered expression is reduced expression.

20. The method of claim 16, wherein ADPKD is associated with reduced or altered activity of PKD1 protein and/or PKD2 protein.

21. The method of claim 16, wherein ADPKD is associated with increased renal epithelial cell proliferation.

22. The method of claim 16, wherein ADPKD is associated with bilateral renal enlargement and cyst.

23. The method of claim 2, wherein treating kidney disease comprises inhibiting AVP production in the subject.

24. The method of claim 3, wherein treating liver disease comprises inhibiting AVP production in the subject.

25. The method of claim 6, wherein treating congestive heart failure comprises inhibiting AVP production in the subject.

26. The method of claim 7, wherein treating cirrhosis of the liver comprises inhibiting AVP production in the subject.

27. The method of claim 8, wherein treating high altitude pulmonary hypertension comprises inhibiting AVP production in the subject.

28. The method of claim 10, wherein treating severe depression comprises inhibiting AVP production in the subject.

29. The method of claim 11, wherein treating autism comprises inhibiting AVP production in the subject.

30. The method of any of claims 23 to 29, wherein inhibiting AVP production lowers AVP plasma level in the subject.

31. The method of claim 30, wherein lowered AVP plasma level in the subject may be detected as a lower urinary AVP concentration or excretion by the subject.

32. The method of claim 2, wherein treating kidney disease comprises slowing down the progression of the disease.

33. The method of claim 2, wherein treating kidney disease comprises slowing down progression of a cystic disease.

34. The method of claim 33 or 34, wherein slowing down progression of the disease or of the cystic disease comprises reducing risk of developing kidney failure and/or end-stage renal disease.

35. The method of claim 2, wherein treating kidney disease comprises reducing cAMP production in renal collecting duct cells of the subject.

36. The method of claim 2, wherein treating kidney disease increases urine output and/or decreases urine osmolality.

37. The method of claim 11, wherein autism is autism spectrum disorder (ASD).

38. The method of claim 35, wherein elevated activity of AVP-V2 receptor-cAMP axis comprises elevated circulating AVP and/or elevated V2 receptor signaling in the subject.

39. The method of claim 38, wherein elevated V2 receptor signaling comprises elevated cAMP level in a V2 receptor positive cell of the subject.

40. The method of claim 39, wherein the V2 receptor positive cell is a renal collecting duct cell.

41. The method of claim 40, wherein the renal collecting duct cell is or comprises a principal cell.

42. The method of claim 41, wherein the principal cell translocates aquaporin protein to apical plasma membrane.

43. The method of claim 35, wherein lowering of cAMP level results in a decrease number of aquaporin proteins on apical surface of the principal cell.

44. The method of claim 35, wherein lowering of cAMP level results in a decreased expression of aquaporin genes.

45. The method of claim 43, wherein the aquaporin proteins are selected from the group consisting of aquaporin protein 2 (AQP2) and aquaporin protein 3 (AQP3).

46. The method of claim 45, wherein the aquaporin protein is aquaporin protein 2 (AQP2).

47. The method of claim 43, wherein the decreased number of aquaporin proteins on apical surface of the principal cell result in a decreased re-absorption of water by the principal cell or renal collecting duct cell, increased urine output, decreased urine osmolality, and/or decreased urinary AVP excretion.

48. The method of claim 47, wherein decreased urinary AVP excretion positively correlates with a decreased plasma AVP level.

49. The method of any of the preceding claims, wherein the subject has been diagnosed with a need for treatment prior to the administering step.

50. The method of any of the preceding claims, further comprising administering one or more additional therapeutic agents.

51. The method of any of the preceding claims, wherein the composition comprising an effective amount of AR-C124910XX or AR-C133913XX is administered orally, intravenously, subcutaneously or intramuscularly, as an implant or patch, or via a needle or microneedles.

52. The method of any of the preceding claims, wherein the step of administering to the subject a composition comprising an effective amount of AR-C124910XX or AR-C133913XX is a long-term treatment regimen, wherein the long-term treatment regimen is at least 2 weeks.
